(19) United States
(12) Patent Application Publication
(10) Pub. No.: US 2015/0141877 A1
(43) Pub. Date: May 21, 2015

(54) LED AND SHOCKWAVE THERAPY FOR TATTOO REMOVAL

(71) Applicant: Stephen E. Feldman, New York, NY (US)

(72) Inventor: Stephen E. Feldman, New York, NY (US)

(73) Assignee: INREXREM INC., New York, NY (US)

(21) Appl. No.: 14/516,235

(22) Filed: Oct. 16, 2014

Related U.S. Application Data
(63) Continuation-in-part of application No. 13/573,624, filed on Sep. 28, 2012.
Application No. 13/573,624 is a continuation-in-part of application No. 12/381,134, filed on Mar. 6, 2009.
(60) Provisional application No. 61/068,369, filed on Mar. 7, 2008.

(51) Int. Cl. A61N 5/06

(52) U.S. Cl. CPC A61N 5/0616; A61N 2005/0663; A61N 2005/0659; A61N 2005/0652
USPC 601/18

(57) ABSTRACT

A tattoo can be removed from a subject using extracorporeal shock waves and light. The extracorporeal shook waves (ESW) can have an energy level of less than 0.27 mJ/mm2 and be administered to an unaltered tattooed region of a subject for approximately 10 minutes. A continuous, non-pulsing light of a wavelength between 400-940 nm having an energy output of about 50,000 Lux from the optical device can then be administered to the tattooed region within approximately two minute after administering the ESW at a distance of approximately 1 to 2 inches above the tattooed region for approximately 5 to 15 minutes. This allows the tattoo to be removed due to molecular vibration and molecular bond deformation which causes the bonds of the tattoo ink to break apart and be dispersed and absorbed into a body of the subject.

This application is a continuation-in-part of U.S. patent application Ser. No. 13/573,624, filed Sep. 28, 2012, now pending, which is a continuation-in-part of U.S. patent application Ser. No. 12/381,134, filed Mar. 6, 2009, now pending, and claims the benefit of provisional application Ser. No. 61/941,173, filed Sep. 26, 2008 and provisional application Ser. No. 61/068,369, filed Mar. 7, 2008. The patent applications identified above are incorporated here by reference in their entirety to provide continuity of disclosure.

### BACKGROUND

The subject matter described herein relates to LED and shockwave therapy for tattoo removal. There are a variety of medical procedures and techniques used to remove tattoos. For example. dermabrasion is one such technique. Dermabrasion slices off or abrades the skin in which the tattoo lies. This procedure is highly invasive and often produces scars. This technique also has a tendency to leave behind pigments which lie in skin layers not removed that appear as a dark shade through the new skin.

Another technique, called a “split-skin graft,” involves the tangential excision of the tattoo area and covers the area with a skin graft. This procedure cuts out the visible tattoo area and leaves intact an underlying skin layer. The procedure is usually performed while a patient is under general anesthesia. The open area is covered with split skin and saved from unnecessary scar formation by use of compression bandages.

Another technique involves the use of lasers and pulsed radiation. These techniques also have many disadvantages. One disadvantage is that the procedure produces “speckles” on the skin due to the high power density of the light beam. The light beam can also cause significant local heating and destruction of tissues that do not contain tattoo ink. To counteract this damage heat must be removed to prevent tissue damage but this wastes a majority of the light beam's power. Another disadvantage is that these procedures involve the use of light that has a short duty cycle and specific wavelength and is thus not absorbed by some colors of tattoo ink. Another disadvantage is that the procedures cannot treat large surface areas and focuses on a very small area. In order for a tattoo to be removed, a patient must undergo many hours of sometimes painful treatment which increases with the size of the tattoo. If a large tattoo is to be removed, the tattoo treatments can be expensive. Also, these light beams can cause reactions in certain chemicals used in the inks leading to permanent darkening.

### SUMMARY

The subject matter described herein relates to LED and shockwave therapy for tattoo removal.

In one implementation, a method of removing a tattoo from a subject using extracorporeal shock waves and light, the method comprising the steps of: generating extracorporeal shock waves (ESW) having an energy level of less than 0.27 mJ/mm2; administering the extracorporeal shock waves to an unaltered tattooed region of a subject for approximately 10 minutes; generating continuous, non-pulsing light of a wavelength between 400-940 nm having an energy output of about 50,000 Lux from the optical device, the optical device having an LED-panel housing a plurality of ultra-bright light emitting diodes (LEDs) in an array that concentrates the energy output; and administering the continuous, non-pulsing light to the tattooed region within approximately two minute after the ESW administering step at a distance of approximately to 2 inches above the tattooed region for approximately 5 to 15 minutes thereby allowing the energy output of the continuous, non-pulsing light to penetrate through an epidermis of the subject and be absorbed into the released tattoo ink, wherein the absorption of the energy output into the released tattoo ink results in the tattoo being removed due to molecular vibration and molecular bond deformation which causes the bonds of the tattoo ink to break apart and be dispersed and absorbed into a body of the subject.

In some implementations, castor oil can be applied to the tattooed region before administering the extracorporeal shock waves, L-Arginine can be applied to the tattooed region before administering the continuous, non-pulsing light or an immune response modifier compound can be applied to the tattooed region before administering the continuous, non-pulsing light. The immune response modifier compound can contain L-Arginine and be selected from the group consisting of: imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydronaphthyridine amine, an oxazoloquinoline amine, a thiazoloquinoline amine, an oxazolopyridine amine, a thiazolopyridine amine, an oxazolonaphthyridine amine, a thiazolonaphthyridine amine, and a 1H-imidazodimer fused to a pyridine amine, a quinoline amine, a tetrahydroquinoline amine, a naphthyridine amine, and a tetrahydronaphthyridine amine.

In another implementation, an apparatus for applying a light and shock wave treatment on a tattooed area of a subject for tattoo removal, the apparatus comprising: an extracorporeal shock wave device, the extracorporeal shock wave device generating low-energy shock waves, the low-energy shock waves being applied to the tattooed area for a first specified period of time resulting in cavitation of tattooed cells; and a light panel housing at least one ultra-bright light emitting diode (LED), the panel producing a continuous light, the at least one ultra-bright LED continuously applying the energy output from the at least one ultra-bright LED directly over the entire tattooed area for a second specified period of time resulting in degradation of the tattoo ink.

In some implementations, the extracorporeal shock wave device can administer the shock waves having energy levels below 0.27 mJ/mm2 for approximately 10 minutes.

In some implementations, the light generated by the at least one ultra-bright LED is approximately equal to size of the tattooed area. The light can have an energy output of about 88 joules per square inch without the use of pulsed radiation and be administered for approximately 5-15 minutes.

In some implementations the extracorporeal shock waves are administered in pulses in order to allow tissue recovery between each pulse.

In another implementation, a method for removing tattoos comprising the steps of: applying an oil to a tattooed skin region: positioning an extracorporeal shock wave device above the tattooed skin region; exposing the tattooed skin region to low-energy shockwaves for a first specified period of time resulting in cavitation of tattooed cells; cooling the tattooed skin region; applying L-arginine to a tattooed skin region, positioning an optical device including at least one LED at a specific distance from said tattooed skin region, and exposing said tattooed skin region to continuous LED energy without pulsing in the range of 400 nm to 940 nm wavelengths for a second specified period of time.

In some implementations, the low-energy extracorporeal shock wave device administers the shock waves with energy levels below 0.27 mJ/mm2 for approximately 10 minutes.

In some implementations, the light penetrates an epidermis of the subject without damaging the epidermis by overheating and enters a dermis of the subject in which tattoo ink resides and results in (a) minimal absorption by melanin and hemoglobin of the subject and (b) little to no heat being generated on the epidermis of the subject while generating heat on the tattoo ink thereby causing increased molecular motion and bond deformation of the tattoo ink.

In some implementations, the light generated by the optical device is approximately equal to size of the tattooed area with an energy output of about 88 joules per square inch without the use of pulsed radiation for approximately 5-15 minutes.

### BRIEF DESCRIPTION OF THE DRAWINGS

FIG. 1 is a perspective view of a light panel constructed in accordance with the disclosed technology;

FIG. 2 is a perspective view of a light panel constructed in accordance with the disclosed technology;

FIG. 3 is a perspective view of a light panel constructed in accordance with the disclosed technology;

FIG. 4 is a block diagram showing various components which are used along with the device constructed in accordance with the disclosed technology;

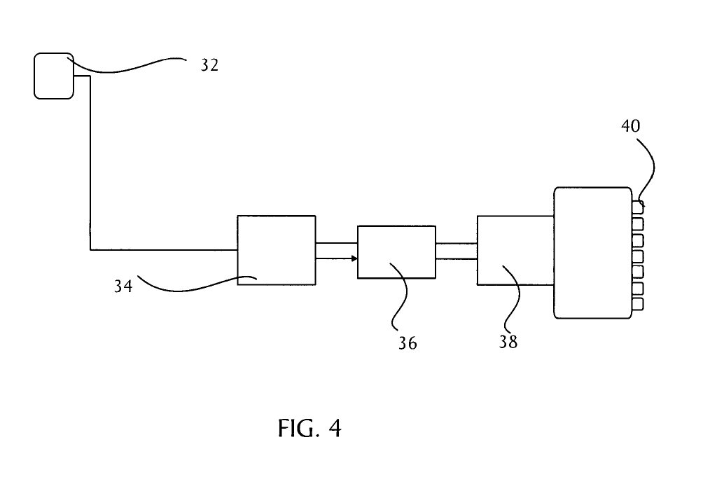

FIG. 5 shows an implementation of the disclosed technology that uses a flexible neck in accordance with the disclosed technology;

FIG. 6 is a perspective view of a light panel constructed in accordance with the disclosed technology

FIG. 7 is a perspective view of a combination ultrasound and light panel constructed in accordance with the disclosed technology; and

FIG. 8 is a perspective view of a combination shockwave and light panel constructed in accordance with the disclosed technology.

### DETAILED DESCRIPTION

Although specific terms are used in the following description for sake of clarity, these terms are intended to refer only to particular structure of the invention selected for illustration in the drawings, and are not intended to define or limit the scope of the invention.

During tattoo applications, a subject's skin cells consume and store tattoo particles. More specifically, tattoo ink contains carbon particles that are suspended in water. When the tattoo ink is introduced to the skin through a needle, the water diffuses. The ink itself then spreads into the surrounding tissue cells and embeds into the skin.

The disclosed technology found using certain energies and wavelengths of light can destroy the bonds that hold tattoo ink together. In operation, a light device uses the energy contained in a light beam so that the energy is absorbed by the tattoo ink dyes. For example, in one implementation, an optical device comprising an LED-panel housing a plurality of light emitting diodes (LEDs) in a tight array can generate continuous, non-pulsing light of a wavelength between 660 nm and 700 nm having an energy output of about 88 joules per square inch by the LEDs of the LED-panel. This light can be administered to an unaltered tattooed region of a subject in a distance of approximately 1 to 2 inches above the tattooed region for approximately 5 to 15 minutes thereby allowing the tattooed region to receive an average energy output of 480 Joules. This light penetrates through an epidermis of the subject and can be absorbed into tattoo ink of the tattooed region residing in a dermal layer of the subject where the absorption of the energy into the tattoo ink results in the tattoo being removed due to molecular vibration and molecular bond deformation which causes the bonds of the tattoo ink to break apart and be dispersed and absorbed into a body of the subject.

For tattoo removal, an ultra-bright LED with high energy output is contemplated. The ultra-bright LED is capable of emitting a pure color in a narrow frequency range, The color emitted from the ultra-bright LED is identified by peak wavelength (lpk) and measured in nanometers (nm). Different LED chip technologies emit light in specific regions of the visible light spectrum and produce different intensity levels. Intensity is a measure of the time-averaged energy flux or amount of light striking a given area for LEDs this is measured in terms of lumens while for a LED lighting apparatus it is measured in lux (lumens/sq. meter). Ultra bright LEDs have a brightness or luminance intensity of 5,000 to 20,000 mcd with a beam angle of 8-130 degrees which equates to a luminance flux of 0.05 to 75 lumen.

LED light output varies with the type of chip, encapsulation, efficiency of individual wafer lots and other variables. The amount of light emitted from an ultra-bright LED is quantified by a single point, on-axis luminous intensity value (lv), LED intensity is specified in terms of millicandela (mod). MCD or Millicandela is used to denote the brightness of an LED. The higher the mcd number, the brighter the light the LED emits. Ultra bright LED's have mcd ratings that vary between 5,000 and 20,000 mcd with beam angles of 8 to 130 degrees.

LED viewing angle is a function of the LED chip type and the epoxy lens that distributes the light. View angle degree, also referred to as directivity, or the directional pattern of a LED light beam is measured in degrees. The expressed degree dictates the width of the light beam and also controls to some extent, the light intensity of a LED. View angles range from 8 to 160 degrees, and are provided through the use of optics, e.g., special lenses made to collimate light into a desired view angle. The highest luminous intensity (mcd rating) does not equate to the highest visibility. The light output from an LED chip is very directional, A higher light output is achieved by concentrating the light in a tight beam. Generally, the higher the mcd rating, the narrower the viewing angle.

Another factor is the ultra-bright LED's wavelength. Nanometers or nm are used to measure the wavelengths of light. The lower the wavelength, e.g., 400 nm, the bluer and stronger the light source. Longer wavelengths above 600 nm are red. Above 680 nm, they fall into the Infra-Red category, which is colorless to our eyes. White LEDs have no specific wavelength. They are measured by the color of white against the chromaticity scale.

The frequency of light used to destabilize the bonds in tattoo inks depends upon the composition of the ink and its color. Additional considerations are absorption by the subject's tissue cells, For example, melanin and hemoglobin have maximum absorptions below 600 nm, i.e., maximum absorption for melanin is 335 nm and for hemoglobin is 310 nm.

In use, the primary wavelength range may be between 400 to 940 nm. The primary wavelengths are carefully chosen so that (a) there is minimal to no absorption by melanin and hemoglobin of a subject and little to no heat is generated on the epidermis of the subject and (b) enough heat is generated so that tattoo ink residing in a dermis of the subject is irradiated sufficiently to cause increased molecular motion and bond deformation of the tattoo ink. It is worthy to note that the disclosed technology does not depend on the photomodulation of living tissue but creates an environment where there is little to no photomodulation of living tissue and a high amount of photomodulation in relation to the bonds of the tattoo ink.

The light beam used in the disclosed technology is generated by an ultra-bright LED(s). The energy output from the ultra-bright LED(s) is concentrated on a tattooed area of the recipient. The energy output generated during a removal session penetrates the epidermis of the recipient and goes through the epidermis into the dermis in which the tattoo ink is situated. The energy output is such that the light degrades the tattoo ink but does not cause any damage the surrounding tissue cells.

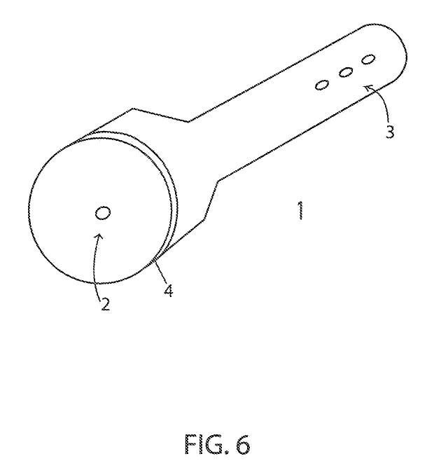

In one implementation, as shown in FIG. 6, a light panel 1 can house a single ultra-bright light emitting diode (LED) 2. The light panel can also have an adjustable lens 4 over the LED 2, The LED 2 can have a primary wavelength between 400-940 nm and produce a continuous and non-pulsed light. The panel 1 can have an LED intensity of about 15,000-20,000 mcd with a viewing angle of 8-30 degrees equating to ˜0.2 to 5 lumen. The light panel 1 can include controls 3 that actuate, deactuate, and regulate the light beam of the ultra-bright LED. The light beam can be directly applied over the entire tattooed area, generating a pre-determined illumination (unit Lux or lx), for a specified period of time (approximately 5-30 minutes) resulting in degradation of the tattoo ink by penetrating an epidermis of the subject without overheating and/or damaging the epidermis and enters a dermis of the subject in which tattoo ink resides.

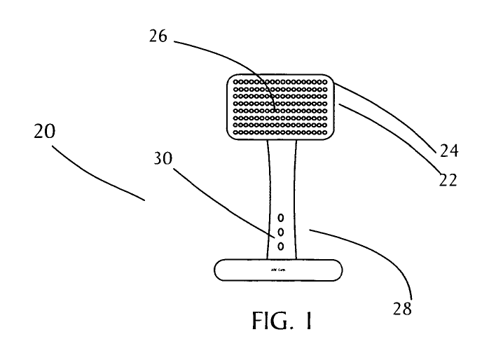

In another implementation, as shown in FIG. 1, the light panel can include a tight array of ultra-bright LEDs having an LED intensity of about 5,000-10,000 mcd with a viewing angle of 30-120 degrees equating to ˜1 to 35 lumen without the use of pulsed radiation. As more LED's are used the intensity can be lowered as well as increasing the size of the beam angle in order to distribute a certain amount of light evenly on the entire tattoo area. The array of LEDs can deliver high intensity light to skin containing tattoo ink. The array also may contain LEDs of several peak intensities to cover a wider visible spectrum output dependent on the colors of the tattoo.

The energy output from the tight array of ultra-bright LEDs can be continuously applied directly over the entire tattooed area, generating a pre-determined illumination (unit Lux or lx), for a specified period of time resulting in degradation of the tattoo ink. The time the tattooed skin is exposed to the light of the ultra-bright LED and the illumination factor is dependent upon factors including the colors in the tattoo as well as the tattoo size.

The light panel 20 can include a proximal end 22 that has an ultra-bright LED panel 24. The ultra-bright LED panel 24 can house one or more ultra-bright LEDs 26. In some embodiments, the device has a distal end 28 that has a control device 30 that has switches to actuate, deactuate, and regulate the ultra-bright LED panel 24. The distal end may be configured so that the LED panel 24 and the plurality of ultra-bright LED cluster probes direct the panel.

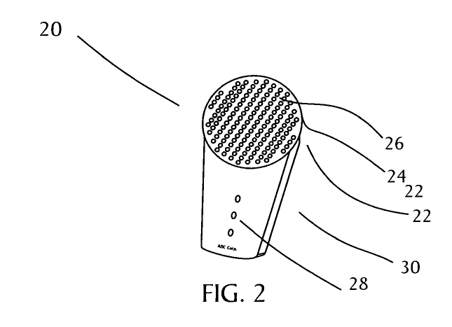

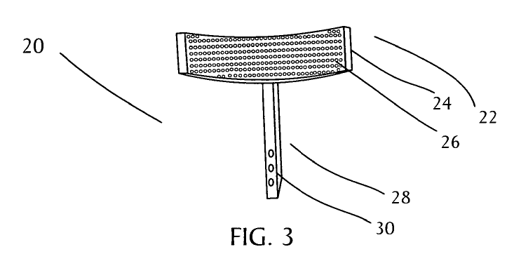

Referring to FIG. 2, a hand-held light panel 20 can be circular in shape. It is, however, understood, that the hand-held light panel 20 may be any different type or shape. Referring to FIG. 3, the LED panel 24 is slightly concave and is designed is for treating facial tattoos, The LED panel 24 may be advantageously shaped for treating facial tattoos of a person who is sitting in a chair.

Referring to FIG, 4, a block diagram shows various components that are used with an optical device constructed in accordance with the present invention are shown, In some implementations, the components are an AC power supply 32 that supplies power to an AC to DC converter 34 that is connected to a timer 36, a PCB (Printed Circuit Board) circuit 38 and ultra-bright LED clusters 40 in series. The AC power supply 32 is converted to DC power supply by the AC to DC converter 34. The timer 36 that is connected in series to the converter 34 controls the time for which the ultra-bright LED clusters 40 is in operation. The PCB circuit 38 is able to provide a variety of time and intensity settings for the timer 38 and ultra-bright LED clusters 40. The time for which the ultra-bright LED clusters are kept on may vary from case to case. Similarly, the intensity of the light produced by ultra-bright LED clusters may vary and the number of ultra-bright LED clusters that are in operation can be changed depending upon the requirement, e.g., size and color of the tattoo. The number of ultra-bright LED dusters that are on is adjusted using the settings provided by the PCB circuit 38. The LED ultra-bright dusters are configured to penetrate the outer skin layer without damaging said outer skin for effective tattoo removal. The average energy output, in a 15 minute session, can be approximately 300-600 Joules. For example, if an LED puts out ˜0.004 W/cm2 at 10 cm distance and 1 W=60J/min in 15 min 3.6 J/cm2 are produce which equates to ˜360 J for a 10×10 cm area. However, it is understood to one skilled in the art that the average energy output can also vary depending on the length of the session and output of the LED(s).

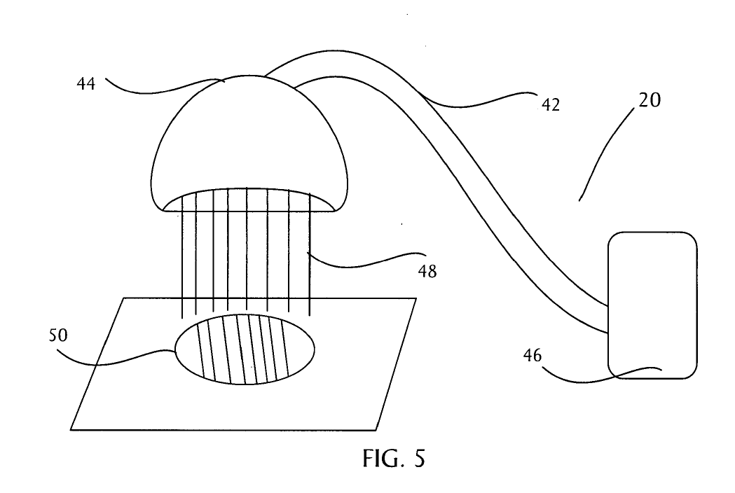

Referring to FIG. 5, FIG. 5 depicts a diagram that illustrates a use of a flexible neck in accordance with the optical device 20 in accordance with the present invention. A flexible neck 42 connects the lamp 44 containing the ultra-bright LED clusters to a power board 46. The flexible neck advantageously allows the device 20 to be maneuvered and focuses the light 48 radiated by the ultra-bright LED clusters on a tattooed area 50 with greater accuracy and flexibility.

In use, L-Arginine can be applied to the tattooed region before administering the LED light to assist in the fading process but is not necessary for the disclosed technology to fade a tattooed area. The L-Arginin helps create enlarged blood vessels which bring greater blood flow to the tattoo area. In addition, it creates an increase in the immune system response. These two mechanisms may help speed up the removal of the by-products of the degradation of the tattoo dyes, thus, allowing for the tattoo to fade more quickly, Additionally, an IRM (immune response modifier) compound can be applied. Specifically, IRM compounds containing L-Arginine can also increase the concentration of macrophages in the blood. Macrophages are specifically located in the lymph nodes and are white blood cells that phagocytizes necrotic cell debris and foreign material, including viruses, bacteria, and tattoo ink. The IRM compound may be selected from a group consisting of imidazoquinollne amine; a tetrahydroimidazoquinoline amine; an imidazopyridine amine; a 1,2-bridged imidazoquinoline amine; a 6,7-fused cycloalkylimiciazopyridine amine; animidazonaphthyridine amine; a tetrahydronaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazodimer fused to a pyridine amine, a quinoline amine, a tetrahydroquinoline amine, a naphthyridine amine, and a tetrahydronaphthyridine amine.

### EXAMPLE 1

The operator places a light apparatus approximately 1 to 2 inches above a small tattooed area. (Please note that when the LED is too close to the subject's skin, e.g., less than 1 inch, the skin can (1) burn after 2-3 sittings (1 sitting=20 min under LED exposure) and/or (2) become rough due to dehydration and change in color intensity of tattooed portion is hardly visible to naked eye whereas when the LED is kept too far from the subject's skin, e.g. more than 2 inches, there is no change in color intensity of tattoo ink.) The light apparatus contains a single Edistar version 9 Warm White LED Product # ENSX-05-0707-EE-1 having 2800 Lumen@2000 mA/300 mA and 25° C., 222.7526 candelas@2000 mA/300 mA and 25° C. with a standard emission angle of 120 degrees. The tattoo area is then exposed to the continuous light generated by the ultra-bright LED for 15 minutes. Depending on the distance, the illumination of the tattooed area is ˜7000 to 30000 lux. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. That is, the energy output of the ultra-bright LED will break apart the bonds of the tattoo ink and cause it to be dispersed and absorbed into the body. By using this energy output, the tattoo can be removed.

### EXAMPLE 2

Apply L-Arginine to a large tattooed region and then place an LED apparatus approximately 1 to 2 inches above the tattooed area. The apparatus can contain 120 Edistar version 9 Cool White LED Product #ENSW-10-1010-EB-1 having 7000 Lumen@2000 mA/300 mA at 25° C., 556.8815 candelas@2000 mA/300 mA at 25° C. with a standard emission angle of 120 degrees clustered in twelve rows of ten LEDs each. Depending on the distance, the illumination of the tattooed area is ˜8000 to 20000 lux per LED. The tattoo area is then exposed to the continuous light generated by the clustered ultra-bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. This treatment can be applied approximately six times over a three to four month period with about two to three weeks between treatments.

### EXAMPLE 3

The operator places a light apparatus approximately 1 to 2 inches above a medium-sized tattooed area. The light apparatus contains 80 Ultra-Bright White 5 mm LED 8000 mcd with viewing angle of 90 degrees clustered in eight rows of ten LEDs each. Depending on the distance, the illumination of the tattooed area is ˜3000 to 12000 lux per LED. The tattoo area is then exposed to the continuous light generated by the clustered ultra-bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

### EXAMPLE 4

The operator applies a thin layer of 10% to 15% of L-Arginine directly to a medium-sized tattoo area. The operator then places a light apparatus approximately 1 to 2 inches above the tattooed area after L-arginine has been administered. The light apparatus contains 100 Ultra-Bright White 5 mm LED 6000 mcd with viewing angle of 100 degrees clustered in ten rows of ten LEDs each. Depending on the distance, the illumination of the tattooed area is ˜6000 to 10,000 lux per LED. The tattoo area is then exposed to the continuous light generated by the clustered ultra-bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

In some implementations, the disclosed technology can be a combination device for applying a treatment of light and ultrasound on a tattooed area of a subject for tattoo removal. The device can include an ultrasound device and a light panel. A control panel controls the plurality of ultra-bright LEDs and ultrasound.

During tattoo applications, dermal cells consume and store tattoo particles in vacuoles in the same manner fat cells store lipids. More specifically, tattoo ink contains carbon particles that are suspended in water. When the tattoo ink is introduced to the skin through a needle, the water diffuses. The ink itself then spreads into the surrounding tissue cells and embeds into the skin. The tattooed cells then adopt an “effective density” analogous to the way fat cells develop a lower density.

In removing the tattoos, it was found that this change in cell density can be used to as advantage. In a process called cavitation, sound waves are used to reduce the pressure of a liquid to the point where tiny bubbles of gas form. When the pressure is raised, the bubble collapses violently, generating huge pressures, albeit on a tiny scale.

Primarily, three key parameters of ultrasound—frequency, intensity, and exposure time—play influential roles in the performance and efficacy of ultrasound-mediated therapies. When used as a tattoo removal technique it was found that high frequency ultrasound at a certain intensity and pulse lengths can be used to target tattooed cells. In a preferred embodiment, an ultrasound device may use a high frequency ultrasound having a minimum frequency of 3 MHz and a maximum frequency of 10 MHz with an intensity of a minimum frequency of 12.0 W/cm2 and maximum of 25.6 W/cm2 because the effects of skin permeability begins to decrease after reaching an intensity of 21.9 W/cm2 or more.

As a result of these multiple factors, the duration of the ultrasound treatment will be modified in order to minimize any potential thermal buildup. Continuous application of ultrasound will not be used. Instead, ultrasound pulses will be implemented in order to allow tissue recovery between each pulse. Furthermore, if necessary, longer intersonication delays can be integrated into the treatment process if thermal buildup develops. Surface cooling can also be used during treatments to minimize thermal injury to the skin, Also recommended is the use of a “spiraling” motion during the treatment process. This is done in order to create a more uniform temperature throughout the treated area. It also decreases the chances of excessive thermal buildup in one specific section.

When using ultrasound, the tattooed cells may be selectively disrupted based on differences in mechanical and acoustic properties between healthy and tattooed cells. That is, different ultrasound frequencies and intensities may be used during the cavitation process to collapse tattoo cells and destroy pigment particles without damaging healthy tattoo-free tissue. The result is a technique that safely, economically, and efficiently removes at least significant portions of the ink. However, ultrasound alone will not remove all of the tattoo ink from the tattooed area.

It was found that if LED light waves where used within a specified time after the application the ultrasound, the ink could be more readily degraded and the body will more quickly rid itself of the tattoo ink. in use, it was also found that using certain wavelengths of light can destroy the bonds that hold tattoo ink together. In operation, the light device works by using the energy contained in the light beam so that the energy is absorbed by the tattoo ink dyes. This absorbed energy results in an increased stretching, vibration and bending of the bonds which hold the dye (ink) molecules together. Ultimately, these bond stresses cause bond deformation with resulting bond failure.

In use, the ultrasound device produces high-frequency ultrasound waves. The high frequency ultrasound waves have a frequency of about 5 MHz and an intensity of about 19.8 W/cm2, The ultrasound sound waves are administered in pulses in order to allow tissue recovery between each pulse. These waves are applied directly to the tattooed area for a specified period of time (approximately 10 minutes) resulting in cavitation of tattooed cells.

After the ultrasound is administered, a light panel housing one or more ultra-bright light emitting diodes (LEDs) having a wavelength between 660-700 nm can be applied to the tattooed region. This application results in (a) minimal absorption by melanin and hemoglobin of the subject and (b) little to no heat being generated on the epidermis of the subject while generating heat on the tattoo ink thereby causing increased molecular motion and bond deformation of the tattoo ink and produces a continuous light. The ultra-bright LED(s) is approximately equal to size of the tattooed area and has an energy output of about 88 joules per square inch without the use of pulsed radiation. The light can be directly applied over the entire tattooed area for a specified period of time (approximately 5-15 minutes) resulting in degradation of the tattoo ink and penetrates an epidermis of the subject without damaging the epidermis by overheating and enters a dermis of the subject in which tattoo ink resides.

### EXAMPLE 5

High frequency ultrasound having a frequency of 5 MHz and an intensity of 19.8 W/cm2 is applied to a tattooed area treated with an ultrasound gel for 10 minutes. The ultrasound will cause cavitation of the tattooed cells. After the ultrasound has been applied, the operator will wipe off the ultrasound gel, wait approximately two minutes for the patient's skin to cool, apply L-Arginine to the tattooed region and then place the LED apparatus approximately 1 to 2 inches above the tattooed area. The apparatus contains one or more ultra-bright LEDs. The tattoo area is then exposed to the continuous light generated by the LED(s) for 15 minutes. The average energy output, in this 15 minute session can be 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. This dual treatment can be applied approximately six times over a three to four month period with about two to three weeks between treatments.

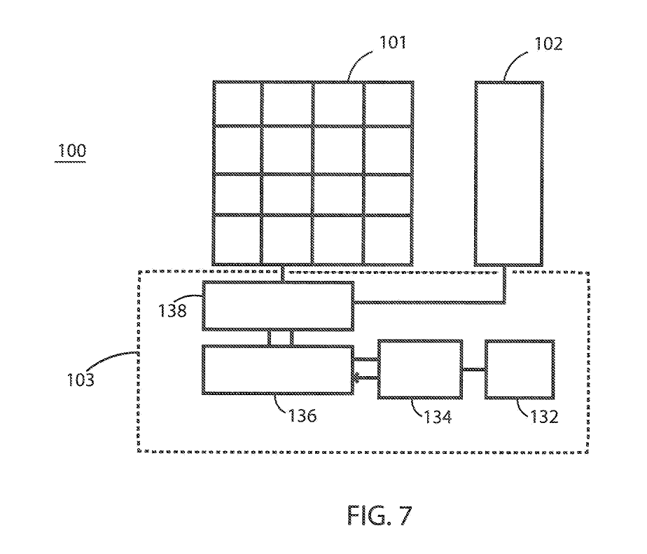

FIG. 7 shows an embodiment of a tattoo removal tool 100 that uses a combination therapy of ultrasound and light. Referring to FIG. 7, a block diagram that shows various components that can be used with a plurality of ultra-bright LEDs 101 and an ultrasound unit 102 constructed in accordance with the disclosed technology are shown. The components of the control panel 103 are an AC power supply 132 that supplies power to an AC to DC converter 134 that is connected to a timer 136, a PCB (Printed Circuit Board) circuit 138. The AC power supply 132 is converted to DC power supply by the AC to DC converter 134. The control panel 103 is capable of controlling plurality of ultra-bright LEDs 101 and an ultrasound unit 102. In some implementations, the timer 186 can be connected in series to the converter 134 for controlling the time for which the plurality of ultra-bright LEDs 101 and ultrasound unit 102 are in operation. That is, the PCB circuit 138 can to provide a variety of time and intensity settings for the plurality of ultra-bright LEDs 101 and ultrasound unit 102. The time for which the plurality of ultra-bright LEDs 101 and the ultrasound unit 102 are kept on may vary from case to case. Similarly, the intensity of the light produced by the plurality of ultra-bright LEDs may vary. Also, the number of LEDs that are in operation can be changed depending upon the requirement and can be adjusted using the settings provided by the PCB circuit 138. In a preferred embodiment, the ultrasound unit can deliver high frequency ultrasound of 5 MHz over discrete time intervals while; the light panel 101 includes a tight array of ultra-bright LEDs having an energy output of about 50,000 Lux without the use of pulsed radiation. The tight array of ultra-bright LEDs 101 continuously applies the energy output from the tight array of ultra-bright LEDs directly over the entire tattooed area for a specified period of time resulting in degradation of the tattoo ink.

The advantages of this combination therapy is that the cavitation causes the tattooed cells to dispel the ink and then once the ink is exposed without the protection of the cell membrane the LED light will further break the bonds of the ink so the body may more readily dispose of the ink naturally.

In another implementation, the disclosed technology can be a combination device for applying a treatment of light and shockwaves on a tattooed area of a subject for tattoo removal. The device can include a shockwave device and a light panel. A control panel controls the plurality of ultra-bright LEDs and shockwave device.

In one implementation, a low energy extracorporeal shock wave treatment (ESWT) can be used instead of ultrasound or in combination with ultrasound to selectively disrupt tattooed cells based on differences in mechanical and acoustic properties between healthy and tattooed cells. ESWT delivers shock waves and sonic pulses with high energy impact which can induce biochemical changes within the targeted tattooed cells through mechanotransduction, The ESWT can be administered before an application of an LED treatment, as described throughout, simultaneously with the application of the LED treatment or after the application of the LED treatment.

True ESWT produces a very strong energy pulse (5-100 MPa) for a very short length of time, (approximately 10 milliseconds). The energy pulse quite literally breaks the sound barrier, and this is what creates the shockwave, The ESWT device can produce a shockwave that is controlled and focused precisely. The ESWT device is capable of controlling and focusing the shockwaves to such an extent that the shockwaves can focused on a treated part of the body and pass through the untreated portions of the body without any effect, and delver the energy to a focus point at the level of the treated tissue.

In use on tattooed cells, the shockwave can exert a mechanical pressure and tension force on the tattooed cells. This has been shown to create an increase in cell membrane permeability, thereby increasing microscopic circulation to the tattooed cells and the metabolism within the tattooed cells. The ESWT shock waves pressure front also creates behind it what are known as “cavitation bubbles”. Cavitation bubbles are simply small empty cavities created behind an energy front. They tend to expand to a maximum size, then collapse, much like a bubble popping. As these bubbles burst, a resultant force is created. In the human body, this force is strong enough to destroy pigment particles without damaging tissue. As cavitation bubbles collapse, they create smaller, secondary energy waves known as microjets. These microjets also create a lot of force that also destroys pigment particles without damaging tissue through direct, mechanical means. In the application of an ESWT treatment, it's not just one cavitation bubble or just a few cavitation bubbles being produced, but hundreds and thousands. Multiply this by several thousand shockwaves being administered to a tattooed region through a course of ESWT treatment.

ESWT treatments can be electrohydraulic, electromagnetic, or piezoelectric technologies. Each technology produces a pulse that literally breaks the speed of sound, thereby creating a shockwave. These technologies differ in the manner in which the shockwaves are produced, the ability of the shockwave to be controlled and focused, the depth to which the shockwaves can penetrate, the intensity of the shockwave being produced. Another therapy, radial therapy, is actually quite different from the other three technologies in several regards and is usually not considered true extracorporeal shockwave therapy—but more of a pressure wave therapy.

Electrohydraulic shockwave therapy uses a type of spark plug to generate a shockwave, with the shockwaves focused by an ellipsoid reflector. Electromagnetic shockwave therapy machines typically use a cylindrical coil arrangement of an electromagnetic generator and a parabolic reflector to focus the shockwaves. The piezoelectric shockwave is generated by an electric pulse, and the shockwave focused by thousands of small crystals in the applicator head. Each of these three technologies is similar in that the shockwaves and force produced in the machines is translated past the skin and superficial tissues without effect, and are instead focused at the desired tissue depth.

The fourth technology, radial shockwave (RSWT) or more accurately, pressure wave therapy, differs from the other forms of shockwave technology in a couple major regards. First, in order for a shockwave to truly be defined as a shockwave, the energy wave must literally be faster than the speed of sound, or 1500 meters per second. This is the speed at which the “shock” of the shockwave is generated, from breaking the sound barrier. In comparison, RSWT waves travel at speeds of approximately 10 meters per second, a small fraction of true shockwave. This speed does not break the sound barrier, and hence, no actual shockwave is produced. Indeed, the very wave form produced by radial technology differs from true shockwave rather noticeably. True focused shockwaves are very short and very intense; radial pressure waves are slower, less intense, elongated, and more sinusoidal in appearance. Because no actual shockwave is produced with RSWT, and because the waveform is so different, you can better see why RSWT is not considered a shockwave technology. It is more accurately described as a pressure wave technology, and most researchers now use this term to describe this technology.

Using this technologies, shockwaves or pressure waves, can be directly aimed at the tattooed region. For example, electrohydraulic, electromagnetic, and piezoelectric shockwave can all be aimed and delivered past the skin and down to different depths, allowing for delivery of the therapeutic waves penetrating through an epidermis of the subject and being absorbed into tattoo ink of the tattooed region residing in a dermal layer of the subject.

The piezoelectric technology is the most accurate ESWT technology. Treatment is more precisely directed at the tattooed region and the least traumatic to tissue surrounding the site being treated. However, because piezoelectric technology is so precise, it needs to be applied carefully and precisely to the correct regions.

Another important differentiating characteristic is how high an energy output the machine produces. For example, when applying the energy things to consider are (1) the amount or type of energy produced by the machine, (2) the amount or type of energy delivered into the body, (3) the amount or type of energy delivered into the focus area, (4) the amount or type of energy delivered to a central point inside the focus area, and (5) the amount or type of energy present at a certain radius from that central focus point.

For the purposes of this discussion, we'll concentrate on the most common standardized measurement of energy in the field, something called the “energy flux density”, expressed in millijoules per millimeter (mJ/mm2). Energy flux density can be defined as the amount or concentration of energy in the focus area. In other words, this is the amount of therapeutic energy being delivered to the tattooed region. For the purposes of this discussion, we'll define low energy here as less than 0.27 mJ/mm2, medium energy as 0.27 mJ/mm2 to 0.59 mJ/mm2, and high energy as anything over 0.60 mJ/mm2. For tattoo removal, the tattooed region appears to respond better to lower energy settings as research indicates that the tattooed region may be damaged by higher-intensity settings.

In one implementation, piezoelectric ESWT can be applied in energy levels as low as 0.05 mJ/mm2—obviously well into the lowest levels of energy—and it can be raised as high as 1.48 mJ/mm2—an energy level well above even the classic “high energy” machines. in other words, in terms of the amount of energy applied in the focus area, (the so-called “energy flux density”), piezoelectric technology can be delivered in energy doses as low as virtually any other competing technology and as high or higher than virtually any other technology. Further, piezoelectric technology can be readily adjusted to any energy level, depending upon the specific condition and indication of each individual case. And as mentioned above, the energy can be precisely focused to the specific depth required.

It was found that if LED light waves where used within a specified time after the application the ultrasound or ESWT, the ink could be more readily degraded and the body will more quickly rid itself of the tattoo ink. It was also found that if light was administered before ESWT, the ink bonds would be broken before cavitation and the ink could be more readily degraded and the body will more quickly rid itself of the tattoo ink once cavitation occurs.

### EXAMPLE 6

Piezoelectric ESWT is applied in energy levels as low as 0.05 mJ/mm2 and applied to a tattooed area treated with a castor oil for 10 minutes. The ESWT will cause cavitation in close proximity to the tattooed cells. After the ESWT has been applied, the operator will wipe off the oil, wait approximately two minutes for the patient's skin to cool, apply L-Arginine to the tattooed region and then place the LED apparatus approximately 1 to 2 inches above the tattooed area. The apparatus contains 120 ultra-bright LEDs 26 clustered in twelve rows of ten LEDs each. The tattoo area is then exposed to the continuous light generated by the clustered ultra-bright LEDs for 15 minutes. The average energy output, in this 15 minute session is 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. This treatment is applied approximately six times over a three to four month period with about two to three weeks between treatments.

The advantages of this combination therapy is that the cavitation causes the tattooed cells to dispel the ink and then once the ink is exposed without the protection of the cell membrane the LED light will further break the bonds of the ink so the body may more readily dispose of the ink naturally.

### EXAMPLE 7

Apply L-Arginine to the tattooed region and then place the LED apparatus approximately 1 to 2 inches above the tattooed area. The apparatus contains 120 ultra-bright LEDs 26 clustered in twelve rows of ten LEDs each. The tattoo area is then exposed to the continuous light generated by the clustered ultra-bright LEDs for 15 minutes. The average energy output, in this 15 minute session is 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Immediately or soon after the light is administered, a piezoelectric ESWT is applied in energy levels as low as 0.05 mJ/mm2 and applied to a tattooed area treated with a castor oil for 10 minutes. The ESWT will cause cavitation in dose proximity to the tattooed cells. This treatment is applied approximately six times over a three to four month period with about two to three weeks between treatments.

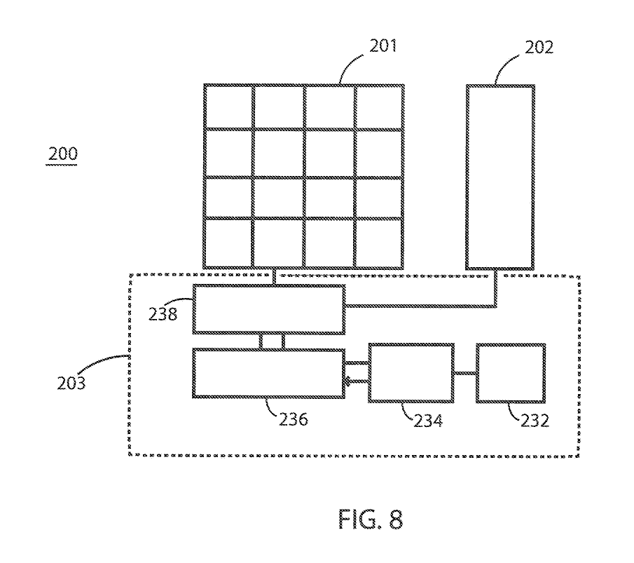

FIG. 8 shows an embodiment of a tattoo removal tool 200 that uses a combination therapy of shockwave therapy and light. Referring to FIG. 8, a block diagram that shows various components that can be used with a plurality of ultra-bright LEDs 201 and a shockwave device 202 constructed in accordance with the disclosed technology are shown. The components of the control panel 203 are an AC power supply 232 that supplies power to an AC to DC converter 234 that is connected to a timer 236, a PCB (Printed Circuit Board) circuit 238. The AC power supply 232 is converted to DC power supply by the AC to DC converter 234. The control panel 203 is capable of controlling a plurality of ultra-bright LEDs 201 and a shockwave device 202. In some implementations, the timer 236 can be connected in series to the converter 234 for controlling the time for which the plurality of ultra-bright LEDs 201 and shockwave device 202 are in operation. That is, the PCB circuit 238 can to provide a variety of time and intensity settings for the plurality of ultra-bright LEDs 201 and shockwave device 202. The time for which the plurality of ultra-bright LEDs 201 and the shockwave device 202 are kept on may vary from case to case. Similarly, the intensity of the light produced by the plurality of ultra-bright LEDs may vary. Also, the number of LEDs that are in operation can be changed depending upon the requirement and can be adjusted using the settings provided by the PCB circuit 238. In a preferred embodiment, the shockwave can deliver energy levels as low as 0.05 mJ/mm2 to 0.027mJ/mm2, the light panel 201 includes a tight array of ultra-bright LEDs having an energy output of about 50,000 Lux without the use of pulsed radiation. The tight array of ultra-bright LEDs 201 continuously applies the energy output from the tight array of ultra-bright LEDs directly over the entire tattooed area for a specified period of time resulting in degradation of the tattoo ink.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention, Those skilled in the art could implement various other feature combinations without departing from the scope and spirit of the invention.

1. A method of removing a tattoo from a subject using extracorporeal shock waves and light, the method comprising the steps of:
generating extracorporeal shock waves (ESW) having an energy level of less than 0.27 mJ/mm2;
administering the extracorporeal shock waves to an unaltered tattooed region of a subject for approximately 10 minutes;
generating continuous, non-pulsing light of a wavelength between 400 940 nm having an energy output of about 50,000 Lux from the optical device, the optical device having an LED-panel housing a plurality of ultra-bright light emitting diodes (LEDs) in an array that concentrates the energy output; and
administering the continuous, non-pulsing light to the tattooed region within approximately two minute after the ESW administering step at a distance of approximately 1 to 2 inches above the tattooed region for approximately 5 to 15 minutes thereby allowing the energy output of the continuous, non-pulsing light to penetrate through an epidermis of the subject and be absorbed into the released tattoo ink, wherein the absorption of the energy output into the released tattoo ink results in the tattoo being removed due to molecular vibration and molecular bond deformation which causes the bonds of the tattoo ink to break apart and be dispersed and absorbed into a body of the subject.

2. The method of claim 1 wherein castor oil is applied to the tattooed region before administering the extracorporeal shock waves.

3. The method of claim 1 wherein L-Arginine is applied to the tattooed region before administering the continuous, non-pulsing light.

4. The method of claim 1 wherein an immune response modifier compound is applied to the tattooed region before administering the continuous, non-pulsing light.

5. The method of claim 1 wherein an immune response modifier compound containing L-Arginine is applied to the tattooed region before administering the continuous, non-pulsing light.

6. The method of claim 4 wherein said immune response modifier is a chemical selected from the group consisting of:
imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydronaphthyridine amine, an oxazoloquinoline amine, a thiazoloquinoline an oxazolopyridine amine, a thiazolopyridine amine, an oxazolonaphthyridine amine, a thiazolonaphthyridine amine, and a 1H-imidazodimer fused to a pyridine amine, a quinoline amine, a tetrahydroquinoline amine, a naphthyridine amine, and a tetrahydronaphthyridine amine.

7. An apparatus for applying a light and shock wave treatment on a tattooed area of a subject for tattoo removal, the apparatus comprising:
an extracorporeal shock wave device, the extracorporeal shock wave device generating low-energy shock waves, the low-energy shock waves being applied to the tattooed area for a first specified period of time resulting in cavitation of tattooed cells; and
a light panel housing at least one ultra-bright light emitting diode (LED), the panel producing a continuous light, the at least one ultra-bright LED continuously applying the energy output from the at least one ultra-bright LED directly over the entire tattooed area for a second specified period of time resulting in degradation of the tattoo ink.

8. The apparatus of claim 7 wherein the extracorporeal shock wave device administers the shock waves having energy levels below 0.27 mJ/mm2.

9. The apparatus of claim 7 wherein the first specified period of time is approximately 10 minutes.

10. The apparatus of claim 7 wherein the light generated by the at least one ultra-bright LED is approximately equal to size of the tattooed area.

11. The apparatus of claim 7 wherein the at least one ultra-bright LED has an energy output of about 88 joules per square inch without the use of pulsed radiation.

12. The apparatus of claim 7 wherein the second specified period of time is approximately 5-15 minutes.

13. The apparatus of claim 7 wherein the extracorporeal shock waves are administered in pulses in order to allow tissue recovery between each pulse.

14. A method for removing tattoos comprising the steps of:
applying an oil to a tattooed skin region;
positioning an extracorporeal shock wave device above the tattooed skin region;
exposing the tattooed skin region to low-energy shockwaves for a first specified period of time resulting in cavitation of tattooed cells;
cooling the tattooed skin region;
applying L-argirine to a tattooed skin region,
positioning an optical device including at least one LED at a specific distance from said tattooed skin region, and
exposing said tattooed skin region to continuous LED energy without pulsing in the range of 400 nm to 940 nm wavelengths for a second specified period of time.

15. The method of claim 14 wherein the low-energy extracorporeal shock wave device administers the shock waves with energy levels below 0.27 mJ/mm2.

16. The method of claim 14 wherein the first specified period of time is approximately 10 minutes.

17. The method of claim 14 wherein the light penetrates an epidermis of the subject without damaging the epidermis by overheating and enters a dermis of the subject in which tattoo ink resides.

18. The method of claim 14 wherein the at least one LED results in (a) minimal absorption by melanin and hemoglobin of the subject and (b) little to no heat being generated on the epidermis of the subject while generating heat on the tattoo ink thereby causing increased molecular motion and bond deformation of the tattoo ink.

19. The method of claim 14 wherein the light generated by the optical device is approximately equal to size of the tattooed area.

20. The method of claim 14 wherein the at least one ultra-bright LED has an energy output of about 88 joules per square inch without the use of pulsed radiation.

21. The method of claim 16 wherein the second specified period of time is approximately 5-15 minutes.
